(19) United States
(12) Patent Application Publication
(10) Pub. No.: US 2013/0018270 A1
(43) Pub. Date: Jan. 17, 2013

(54) OPTICAL OBSERVATION SYSTEM AND METHOD OF OBSERVING EXAMINATION SUBJECT

(71) Applicants: Masaru MIZUNAKA, Tokyo (JP); Masanori MURAYAMA, Wako-shi (JP); Atsushi MIYAWAKI, Wako-shi (JP)

(72) Inventors: Masaru MIZUNAKA, Tokyo (JP); Masanori MURAYAMA, Wako-shi (JP); Atsushi MIYAWAKI, Wako-shi (JP)

(73) Assignees: RIKEN, Wako-shi (JP); OLYMPUS CORPORATION, Tokyo (JP)

(21) Appl. No.: 13/545,276

(22) Filed: Jul. 10, 2012

(30) Foreign Application Priority Data
Jul. 12, 2011 (JP) 2011-154172

(51) Int. Cl. A61B 6/00

(52) U.S. Cl. 600/476

(57) ABSTRACT

Provided is an optical observation system that is provided with a portable device, which is secured to an examination subject A and is moved together with the movement of the examination subject, and an external device disposed away from the examination subject, wherein the portable device is provided with a light source; an illumination optical system radiates light coming from the light source onto an observation target site in the examination subject; a detection optical system optically guides light coming from the observation target site; a photodetector detects the light optically guided thereto by the detection optical system and converts the light to an electrical signal; and a transmitter wirelessly transmits the electrical signal output from the photodetector; and the external device is provided with a receiver receives the electrical signal transmitted from the portable device and a signal processor processes the electrical signal received by the receiver.

### CROSS-REFERENCE TO RELATED APPLICATIONS

This application is based on Japanese Patent Application No. 2011-154172, the contents of which are incorporated herein by reference.

### TECHNICAL FIELD

The present invention relates to an optical observation system and a method of observing an examination subject.

### BACKGROUND ART

In the related art, there is a known observation system that detects radiation emitted from a small animal, such as a mouse or the like, as a potential signal and that wirelessly transmits the detected potential signal to an analysis device (for example, see Patent Literature 1).

### CITATION LIST

### Patent Literature

{PTL 1} U.S. Patent Application, Publication No. 2010/0298700, Specification

### SUMMARY OF INVENTION

### Technical Problem

However, brain activity of a mouse or the like is optically detectable, and it cannot be observed with the observation system of Patent Literature 1, which detects potential signals.

The present invention has been conceived in light of the above-described circumstances, and an object thereof is to provide an optical observation system and a method of observing an examination subject, with which brain activity of or the like of a small animal, such as a mouse, can be optically observed while allowing free movement of the small animal.

### Solution to Problem

A first aspect of the present invention provides an optical observation system comprising a portable device that is secured to an examination subject and is transportable as the examination subject moves; and an external device that is disposed away from the examination subject, wherein the portable device is provided with a light source, an illumination optical system that radiates light coming from the light source onto an observation target site in the examination subject, a detection optical system that optically guides light coming from the observation target site, a photodetector that detects the light optically guided thereto by the detection optical system and converts the light to an electrical signal, and a transmitter that wirelessly transmits the electrical signal output from the photodetector; and the external device is provided with a receiver that receives the electrical signal transmitted from the portable device and a signal processor that processes the electrical signal received by the receiver.

In the first aspect of the present invention, the illumination optical system may be provided with a collector lens that collects the light coming from the light source and an objective lens that focuses the light collected by the collector lens to irradiate the observation target site, and the collector lens and the objective lens may be formed of GRIN lenses or aspheric lenses.

In the first aspect of the present invention may be provided with a relay lens that relays the light coming from the light source, which is collected by the collector lens, to a pupil of the objective lens, and the relay lens may be formed of a GRIN lens or an aspheric lens.

In the first aspect of the present invention, the detection optical system may be provided with a relay lens that relays the light coming from the observation target site, which is collected by the objective lens, to the photodetector, and the relay lens may be formed of a GRIN lens or an aspheric lens.

A second aspect of the present invention provides a method of observing an examination subject comprising radiating light coming from a light source in a portable device on to an observation target site in an examination subject, the portable device secured to the examination subject and transportable as the examination subject moves; detecting, with the portable device, light coming from the observation target site and converting the light to an electrical signal; wirelessly transmitting, with the portable device, information about the detected light converted to the electrical signal; and receiving, with an external device disposed away from the examination subject, the electrical signal transmitted from the portable device and processing the electrical signal.

In the second aspect, the portable device may be provided with an objective lens from which the light coming from the light source emerges and which also collects the light coming from the observation target site, and the objective lens may be provided with a sharp tip which pierces the observation target site.

In the second aspect, the observation target site may be the brain of the examination subject.

In the second aspect, the light coming from the observation target site may be fluorescence or luminescence.

### BRIEF DESCRIPTION OF DRAWINGS

FIG. 1 is a diagram showing the overall configuration of an optical observation system according to an embodiment of the present invention.

FIG. 2 is a diagram showing optical components inside a device main unit of the optical observation system in FIG. 1.

### DESCRIPTION OF EMBODIMENT

An optical observation system 1 according to an embodiment of the present invention will be described below with reference to the drawings.

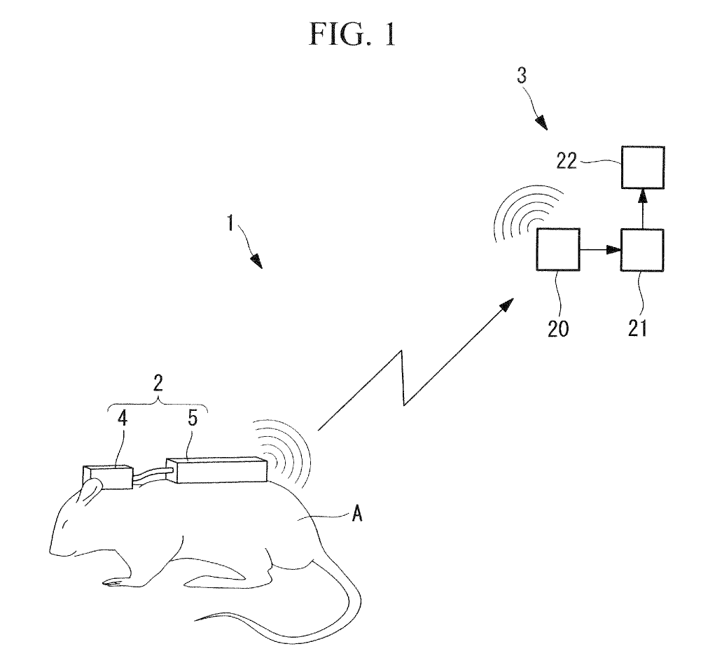

As shown in FIG. 1, the optical observation system 1 according to this embodiment is provided with a portable device 2 that is secured to an examination subject A, such as a small animal like a mouse, etc., and an external device 3 that is disposed at a position distant from the examination subject A.

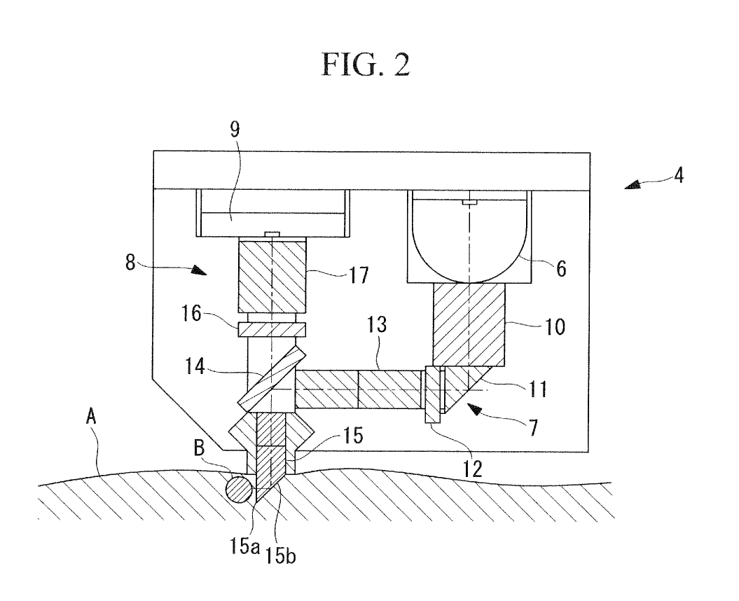

As shown in FIG. 1, the portable device 2 is provided with a device main unit 4 that is secured to the head of the examination subject A and a transmitting unit 5 that is secured to the body thereof. As shown in FIG. 2, the device main unit 4 is provided with a light source 6, such as an LED or the like that emits LED light, an illumination optical system 7 that radiates the LED light coming from the light source 6 onto an observation target site B, a detection optical system 8 that optically guides fluorescence generated at the observation target site B, and a photodetector 9 that detects the fluorescence optically guided thereto by the detection optical system 8.

By the configuration, once the portable device is secured to the examination subject and light emitted from the light source provided in the portable device is radiated onto the observation target site in the examination subject by means of the illumination optical system, light returning from the observation target site is optically guided by the detection optical system and is detected by the photodetector to be converted to an electrical signal. Then, the electrical signal is transmitted by the transmitter provided in the portable device and is received by the receiver of the external device disposed away from the examination subject. Then, the electrical signal received by the receiver is processed by the signal processor. By doing so, information optically detected by the portable device secured to the examination subject can be processed at a location distant from the examination subject. Therefore, brain activity or the like of an examination subject, such as a mouse, can be optically observed while allowing free movement of the examination subject.

The illumination optical system 7 is provided with a collector lens 10 that collects the LED light emitted from the light source 6, a prism 11 that bends the optical path at 90°, an excitation filter 12 that allows only LED light in a predetermined excitation wavelength band included in the LED light to pass through, a relay lens 13 that relays the LED light that has passed through the excitation filter 12, a dichroic mirror 14 that reflects the LED light relayed by the relay lens 13, deflecting it at 90°, while allowing the fluorescence to pass through, and an objective lens 15 that focuses the LED light reflected by the dichroic mirror 14 on the observation target site B. The relay lens 13 relays the LED light emitted from the light source 6, which is collected by the collector lens 10, to a pupil of the objective lens 15.

By the configuration, the light coming from the light source is collected by the collector lens and is efficiently radiated onto the observation target site via the objective lens. Further, the light coming from the light source, which is collected by the collector lens, is relayed by the relay lens, which makes it possible to keep the beam diameter small. Accordingly, point illumination can be employed while preventing vignetting of the beam.

The collector lens 10, the relay lens 13, and the objective lens 15 are formed of GRIN lenses. The objective lens 15 has a sharp tip 15a that can easily pierce the examination subject A (for example, brain tissue) and also has a deflection surface 15b that laterally emits the LED light by deflecting it at 90°.

By forming the collector lens and the objective lens with GRIN lenses or aspheric lenses, the illumination optical system can be made compact as a whole, the portable device, which is moved together with the examination subject by being mounted thereon, can be made lightweight, and thus, optical observation can be performed without hindering the movement of the examination subject.

Further, by forming this relay lens with a GRIN lens or an aspheric lens, the illumination optical system can be made compact as a whole, the portable device, which is moved together with the examination subject by being mounted thereon, can be made lightweight, and thus, optical observation can be performed without hindering the movement of the examination subject.

The detection optical system 8 is formed of a barrier filter 16 that blocks light in the excitation wavelength band included in the fluorescence, which is collected by the objective lens 15 and has passed through the dichroic mirror 14, thus allowing only the fluorescence to pass through, and a relay lens 17 that relays the fluorescence that has passed through the barrier filter 16. The relay lens 17 is also formed of a GRIN lens.

By the configuration, the light emitted from the observation target site is collected by the objective lens, after which the light is relayed by the relay lens and is detected by the photodetector. By forming this relay lens with a GRIN lens or an aspheric lens, the detection optical system can be made compact as a whole, the portable device, which is moved together with the examination subject by being mounted thereon, can be made lightweight, and thus, optical observation can be performed without hindering the movement of the examination subject.

The photodetector 9 is, for example, a photodiode, and is configured to output an electrical signal according to the intensity of received fluorescence.

The transmitting unit 5 is provided with, for example, a transmitter (not shown) that wirelessly transmits the electrical signal output from the photodetector 9 to the exterior and a battery (not shown) that supplies power to the entire portable device 2.

The external device 3 is provided with a receiver 20 that receives the electrical signal transmitted from the portable device 2 by means of the transmitter, a signal processor 21 that processes the electrical signal received by the receiver 20, and a monitor 22. The signal processor 21 performs imaging processing for generating an image from the electrical signal and display processing for displaying the generated image on the monitor 22.

The operation of the thus-configured optical observation system according to this embodiment will be described below.

To optically observe the examination subject A, such as a mouse or the like, by employing the optical observation system 1 according to this embodiment, the sharp tip 15a of the objective lens 15 provided in the device main unit 4 of the portable device 2 pierces an observation target, for example, the brain of the examination subject A, thus securing the device main unit 4 to the head of the examination subject A. The transmitting unit 5, which is relatively heavy, including the battery, is secured to the body of the examination subject A with a belt or the like.

Then, once the light source 6 is activated and the LED light is emitted, the emitted LED light is collected by the collector lens 10 and is deflected by the prism 11; the LED light in the excitation wavelength band that has passed through the excitation filter 12 is relayed by the relay lens 13; and, subsequently, the light is reflected at the dichroic mirror 14 to be radiated onto the observation target site B via the objective lens 15. Because the objective lens 15 has the deflection surface 15b, the LED light is deflected at 90° and is radiated onto the observation target site B located laterally with respect to the direction in which the objective lens 15 pierces the observation target site B.

The fluorescence generated at the observation target site B is collected by the objective lens 15 and passes through the dichroic mirror 14; the LED light from which the light in the excitation wavelength band has been removed by the barrier filter 16 is relayed by the relay lens 17, and then the light is detected by the photodetector 9. The photodetector 9 outputs an electrical signal having intensity according to the intensity of the fluorescence, which is transmitted to the transmitting unit 5.

At the transmitting unit 5, the electrical signal transmitted from the device main unit 4 is wirelessly transmitted to the exterior by means of the transmitter.

At the external device 3, the electrical signal transmitted from the transmitter is received and is turned into an image by the signal processor 21, and the image is then displayed on the monitor 22.

With the thus-configured optical observation system 1 according to this embodiment, because the device main unit 4 mounted on the examination subject A detects the fluorescence generated by radiating the LED light onto the observation target site B, converts it to an electrical signal, and outputs the electrical signal to be wirelessly transmitted to the exterior, an advantage is afforded in that the state of the observation target site B can be optically observed while allowing the examination subject A to freely act without restraining its movement. For example, an advantage is afforded in that brain activity or the like of a small animal, such as a mouse, can be optically observed while allowing free movement of the small animal.

In this case, with optical observation system 1 according to this embodiment, because the collector lens 10, the relay lens 13 and 17, and the objective lens 15 are formed of GRIN lenses, the illumination optical system 7 and the detection optical system 8 can be configured compactly, the portable device 2 can thus be made small and lightweight. Consequently, it is possible to reduce the burden on the examination subject A, it is possible to prevent hindrance to the activities thereof, and thus, it is possible to perform the observation in a freely active state with little stress.

Note that, although the collector lens 10, the relay lenses 13 and 17, and the objective lens 15 of this embodiment are formed of GRIN lenses, alternatively, they may be formed of aspheric lenses. Further, although disposing the relay lens 13 in the illumination optical system 7 enables a point illumination in which an illumination area is made small relative to the observation target site B, alternatively, wide-area illumination may be used without employing the relay lens 13.

Although fluorescence generated at an observation target site is detected in this embodiment, luminescence from reporter molecules, for example, luciferase or the like, may be detected.

Although the embodiment of the present invention has been described in detail as above with reference to the drawings, specific configurations are not limited to this embodiment, and design alterations, etc., within a range that does not depart from the spirit of the present invention are also encompassed.

### REFERENCE SIGNS LIST

A examination subject
B observation target site
1 optical observation system
2 portable device
3 external device
5 transmitting unit (transmitter)
6 light source
7 illumination optical system
8 detection optical system
9 photodetector
10 collector lens
13 relay lens
15 objective lens
17 relay lens
20 receiver
21 signal processor

1. An optical observation system comprising:
a portable device that is secured to an examination subject and is transportable as the examination subject moves; and
an external device that is disposed away from the examination subject,
wherein the portable device is provided with a light source, an illumination optical system that radiates light coming from the light source onto an observation target site in the examination subject, a detection optical system that optically guides light coming from the observation target site, a photodetector that detects the light optically guided thereto by the detection optical system and converts the light to an electrical signal, and a transmitter that wirelessly transmits the electrical signal output from the photodetector; and
the external device is provided with a receiver that receives the electrical signal transmitted from the portable device and a signal processor that processes the electrical signal received by the receiver.

2. An optical observation system according to claim 1, wherein the illumination optical system is provided with a collector lens that collects the light coming from the light source and an objective lens that focuses the light collected by the collector lens to irradiate the observation target site,
wherein the collector lens and the objective lens are formed of GRIN lenses or aspheric lenses.

3. An optical observation system according to claim 2, further comprising:
a relay lens that relays the light coming from the light source, which is collected by the collector lens, to a pupil of the objective lens,
wherein the relay lens is formed of a GRIN lens or an aspheric lens.

4. An optical observation system according to claim 1, wherein the detection optical system is provided with a relay lens that relays the light coming from the observation target site, which is collected by the objective lens, to the photodetector,
wherein the relay lens is formed of a GRIN lens or an aspheric lens.

5. A method of observing an examination subject comprising:
radiating light coming from a light source in a portable device on to an observation target site in an examination subject, the portable device secured to the examination subject and transportable as the examination subject moves;
detecting, with the portable device, light coming from the observation target site and converting the light to an electrical signal;
wirelessly transmitting, with the portable device, information about the detected light converted to the electrical signal; and
receiving, with an external device disposed away from the examination subject, the electrical signal transmitted from the portable device and processing the electrical signal.

6. A method of observing an examination subject according to claim 5, wherein the portable device is provided with an objective lens from which the light coming from the light source emerges and which also collects the light coming from the observation target site,
wherein the objective lens is provided with a sharp tip which pierces the observation target site.

7. A method of observing an examination subject according to claim 5, wherein the observation target site is the brain of the examination subject.

8. A method of observing an examination subject according to claim 5, wherein the light coming from the observation target site is fluorescence or luminescence.
